(19) United States
(12) Patent Application Publication
(10) Pub. No.: US 2021/0244371 A1
(43) Pub. Date: Aug. 12, 2021

(54) CORONARY VEIN GUIDING SYSTEM AND ACCOMPANYING VEIN GUIDING METHOD AND SYSTEM

(71) Applicants: Beijing Chest Hospital, Capital Medical University, Beijing (CN); Beijing Tuberculosis and Thoracic Tumor Research Institute, Beijing (CN)

(72) Inventor: Jian ZHANG, Beijing (CN)

(21) Appl. No.: 17/244,479

(22) Filed: Apr. 29, 2021

Related U.S. Application Data
(63) Continuation-in-part of application No. 15/948,673, filed on Apr. 9, 2018, now abandoned.

(30) Foreign Application Priority Data
Sep. 4, 2017 (CN) 201710785387.X

(51) Int. Cl. A61B 6/12; A61B 8/12; A61B 6/00; A61B 8/08; A61B 8/06

(52) U.S. Cl. CPC A61B 6/12; A61B 8/12; A61B 6/481; A61B 6/504; A61B 6/507; A61B 6/487; A61M 2025/0166; A61B 6/467; A61B 8/488; A61B 8/06; A61B 8/481; A61B 6/4417; A61B 6/541

(57) ABSTRACT

A coronary vein guiding system comprises a coronary vein guiding catheter, and a flexible inner tube sheathed therein, comprising a coaxial section, an anti-bending section and a torsion control section; an end portion of the coaxial section being provided with a flexible head end; the coaxial section being connected with the anti-bending section at obtuse angle of 136°-140°; the anti-bending section being connected with the torsion control section at obtuse angle of 138°-142°; the coaxial section and the torsion control section being situated on a same side of the anti-bending section, and the three sections being located on a same plane; the flexible inner tube being 18-22 cm longer than the coronary vein guiding catheter; a head end of the flexible inner tube being in same direction as the coaxial section, a tail end of the flexible inner tube being provided with a screw joint.

### CROSS-REFERENCE TO RELATED APPLICATIONS

This application is a continuation-in-part (CIP) application of U.S. patent application Ser. No. 15/948,673, entitled “Coronary Vein Guiding System”.

### TECHNICAL FIELD

The present disclosure relates to the field of medical instruments, and in particular to a coronary vein guiding system and an accompanying vein guiding method and system.

### BACKGROUND ART

Since a new era of interventional cardiology was opened up by doctor Gruentzig carrying out the first percutaneous transluminal coronary angioplasty (PTCA) in the world in 1977, during the development process of more than thirty years, percutaneous coronary intervention (PCI) has experienced the stages of simple balloon dilation and bare metal stent (BMS) implantation and has developed into the modern drug-eluting stent age. More and more complicated coronary artery diseases have been listed as the indications. As one of the indications of percutaneous coronary intervention, chronic total occlusion (CTO) has a low operation success rate, relatively complex operations, relatively high complications and poor long-term prognosis due to its special pathological and anatomical features, compared with non-lesion operations, and is still the last difficulty that has not been overcome in the field of cardiovascular intervention. Compared with the non-CTO lesions, the CTO lesions are not ideal in both the success rate and the therapeutic effect.

The CTO lesion is a lesion that no antegrade flow is seen when coronary artery is radiographed, with the occlusion having lasted for more than three months, which is referred to as true total occlusion. The main cause is atherosclerotic plaque occlusion. At present, among people that are demonstrated, by coronary arteriography, to suffer from coronary heart disease, the CTO incidence is about 33%, which is increased along with the age, wherein percutaneous coronary intervention (PCI) is suitable for about 46% of the patients. The NHLBI Registry research results show that the right coronary artery occlusion is more common, then comes the left anterior descending coronary artery. All the CTO lesions develop from the earliest acute event. Due to the rupture of atherosclerotic plaques in the acute stage, thrombi lead to occluded vascular lumina; and as time lapses, the plaques rich in cholesterol lipid are gradually replaced by collagen, with which the organized thrombus structure is mixed into a fibrous structure, and in some lesions, calcification even occurs, thereby gradually developing a fiber calcification occlusion structure with loose and dense connective tissues.

Cardiac interventional therapy is one of the direct and effective approaches for CTO, but compared with non-chronic total occlusions, the operation success rate of the CTO lesions is relatively lower. In the middle of 1980s, the operation success rate was only 48% to 76%, but with the improvement of the experience of the operators, the development of interventional instruments and the emergence of new interventional therapies in recent years, the operation success rate of the chronic total occlusions has been increased to 65% to 92% since the late 1990s, which, however, is still far lower than that of the non-CTO lesions.

The instrument selection is a common cause for the failure of the CTO interventional therapy, and among the failures caused by instrument selection, 85% result from the failure of a guide wire in passing through the occluded lesion, followed by the failure of a balloon in passing through the occluded lesion (about 10%) or in dilation (about 5%).

Intravascular ultrasound (IVUS) is new means of diagnosis which has been applied to clinical diagnosis of vascular lesions since the 1990s, and the instrument thereof comprises two parts: a catheter with a micro-probe, and an ultrasonic imaging device. IVUS detects, using the ultrasound principle, the structure of the tissues inside the blood vessels, and the tissues of the vascular walls and surrounding the blood vessels, and is an invasive tomography technology guiding disease diagnosis and treatment. As an intravascular iconographic detection technology, IVUS increasingly demonstrates its superiority in the field of percutaneous coronary intervention, which allows for 360° real-time observation of the vascular walls from the inside, and has a resolution of 100 μm, with a projection depth of 4 mm to 8 mm and a scanning range of 10 mm to 15 mm. Since IVUS makes use of the phenomenon of reflection of the sound waves, it is beneficial to the display of the deep structure. In percutaneous coronary intervention, IVUS can accurately reflect the nature, the order of severity and the accumulation range of coronary artery lesions, and the diameter of the referenced blood vessels, and can therefore guide the operator to select correct strategies to treat the lesions, and help the operator to select a stent with a proper size. Moreover, IVUS can be used for evaluating the effect of coronary stent operation, which helps the operator to find and solve the problems in time after the stent has been implanted, so as to achieve the optimum effects of interventional therapy. Thus, compared with coronary interventional treatment under the guidance of coronary arteriography, the IVUS technology can further optimize the effects of coronary interventional treatment. By applying IVUS to intervention operations for coronary CTO lesions, the operation success rate and the overall technical level of the CTO intervention can be improved. In the mid-period of 1980s to 1990s, the intervention operation success rate was 48% to 76% in the world, and the success rate of the CTO-lesion intervention operations has been increased to 65% to 92% since the late 1990s; and in China, for the experts, the intervention operation success rate at present is 70% to 90%.

Due to the complexity of the structure of heart, among the existing CTO cases, very few collateral vessels meet the IVUS catheter application conditions, and the IVUS catheter cannot achieve guiding in real time the working guide wire to travel in an occlusion of the blood vessels. Moreover, due to the long learning curve and high technical requirement on the operator, the effects of using IVUS for guiding are limited, and the operation success rate is poor.

Moreover, in the actual operation process, because the IVUS catheter cannot guide, in real time, the working guide wire to travel in an occlusion of the blood vessels, the guide wire is likely to enter the vascular dissection (or interlayer) and hence can hardly be returned to the true lumen, which even causes a failure of the operation, thereby greatly increasing the risk and uncertainty of the operation.

Furthermore, a large amount of a contrast agent (300 to 800 ml) is required in a conventional operation. For patients with renal insufficiency, the amount of the contrast agent used should be controlled within 100 ml, but a conventional operation cannot meet the requirement. Even for patients with other body functions being normal, excessive contrast agent will cause burden on the liver and kidney functions of patients. Some patients may even be allergic to the contrast agent and may experience polypnea in severe cases.

In addition, the conventional operation methods are time-consuming, and a large dose of radiation is generated in the operation, thus medical staff has to work intensively and suffer from considerable radiation damage. Furthermore, the long operation time also poses a certain risk to the patient.

In view of the above problems, the present disclosure is proposed.

### SUMMARY

The present disclosure provides a coronary vein guiding system in order to solve the above problems.

In order to achieve at least one of the above objects of the present disclosure, the following technical solution is employed.

The present disclosure relates to a coronary vein guiding system, comprising a coronary vein guiding catheter, and a flexible inner tube sheathed into the coronary vein guiding catheter;

the coronary vein guiding catheter sequentially comprising a coaxial section, an anti-bending section and a torsion control section;

an end portion of the coaxial section being provided with a flexible head end;

the coaxial section being connected with the anti-bending section at an obtuse angle of 136° to 140°, the anti-bending section being connected with the torsion control section at an obtuse angle of 138° to 142°;

the coaxial section and the torsion control section being situated on a same side of the anti-bending section, and the coaxial section, the anti-bending section and the torsion control section being located on a same plane;

the flexible inner tube being 18 cm to 22 cm longer than the coronary vein guiding catheter; and

a head end of the flexible inner tube being in the same direction as the coaxial section, and a tail end of the flexible inner tube being provided with a screw joint.

The coronary vein guiding catheter provided by the present disclosure is provided with two bending angles according to the anatomical features of heart so as to facilitate the insertion into the coronary sinus, wherein the first angle of 137° to 139° helps the coronary vein guiding catheter enter the coronary vein, and the second angle of 139° to 141° enables the coronary vein guiding catheter to be fixed in the coronary sinus and less prone to dislocation.

Only the flexible head end of the coronary vein guiding catheter enters the coronary sinus, the flexible inner tube can freely pass through the coronary vein guiding catheter, with its head end extending from the flexible head end into the venous sinus, so as to establish a safe channel in the great cardiac vein to ensure the operation safety.

After a transfer track is established by placing the coronary vein guiding catheter into the coronary sinus, an ultrasound catheter having ultrasound or other scanning imaging functions is conveyed to the great cardiac vein, and is located at occlusions in the anterior descending branch or the circumflex branch; by collecting information and uploading the same to a workstation, a three-dimensional image of the CTO lesion is reconstructed to guide the intracoronary guide wire to travel in a true lumen in real time, within the CTO occlusion; and the catheter ultrasound is conveyed in the flexible inner tube to the designated site, which, on the one hand, can protect the probe of the catheter ultrasound, and on the other hand, can prevent the ultrasound catheter from scratching heart tissues to cause serious adverse consequences such as pericardial effusion.

In addition, the coronary vein guiding system provided by the present disclosure can further be used for establishing a drug delivery channel so as to inject drugs into heart, or draw blood through the channel established by the coronary vein guiding system.

When the ultrasound catheter and the blood drawing or drug delivery device reach the designated position, the inner tube can be withdrawn.

Preferably, in the coronary vein guiding system described above, the head end of the flexible inner tube is a blunt head end.

Preferably, the flexible inner tube has a two-layered tube wall, with an inner layer being a polyvinyl pyrrolidone (PVP) hydrophilic coating and an outer layer being a high-density polyethylene (HDPE) coating.

Preferably, the screw joint is made of a PC material.

Preferably, in the coronary vein guiding system described above, the coronary vein guiding catheter comprises, from inside to outside, a smooth surface layer, an elastic steel wire layer and a flexible outer layer in sequence.

Preferably, the flexible head end comprises only the flexible outer layer.

Preferably, in the coronary vein guiding system described above, the smooth surface layer is a nylon polytetrafluoroethylene coating.

Preferably, in the coronary vein guiding system described above, the elastic steel wire layer is composed of meshy woven steel wires.

Preferably, the elastic steel wire layer is composed of 12 to 16 layers of steel wires.

Preferably, the steel wire is a 304 stainless steel wire.

Preferably, in the coronary vein guiding system described above, the flexible outer layer is made of a material comprising one or more selected from the group consisting of nylon, polyethylene, silica gel, polymethyl methacrylate, polyvinyl chloride, polytetrafluoroethylene, polyurethane, polyester polymers and silicone.

Preferably, the flexible outer layer is made of a nylon elastomer PEBAX.

Preferably, in the coronary vein guiding system described above, the inner diameter of the coronary vein guiding catheter is 1.5 mm to 1.9 mm, and the thickness of the catheter wall is 0.15 mm to 0.35 mm.

Preferably, the inner diameter of the flexible inner tube is 1.25 mm to 1.4 mm, and the thickness of the tube wall of the flexible inner tube is 0.1 mm to 0.2 mm.

Preferably, in the coronary vein guiding system described above, the length of the coaxial section is smaller than the inner diameter of the right atrium of the inserted heart.

Preferably, in the coronary vein guiding system described above, the length of the anti-bending section is smaller than the distance from the right atrium of the inserted heart to the coronary sinus; more preferably, the length of the anti-bending section is 70 mm to 80 mm.

Preferably, in the coronary vein guiding system described above, the length of the flexible head end is 8 mm to 12 mm.

Compared with the prior art, the advantageous effects of the present disclosure are:

having a simple structure and being adaptive to the anatomical structure of heart, being capable of greatly reducing the implementation difficulty of the heart CTO intervention operation; and being able to be used for drug delivery to heart or blood drawing.

The object of the present disclosure further includes, for example, providing an accompanying vein guiding method and system so as to solve the technical problems existing in the prior art in which the guide wire is likely to enter the vascular dissection and can hardly be returned to the true lumen, a large amount of a contrast agent is required to cause severe side effects to a patient, and an excessively long time and a large dose of radiation are generated in the operation.

In order to achieve the above object of the present disclosure, the following technical solution is employed.

The present disclosure provides an accompanying vein guiding method, comprising the steps of:

S1: acquiring information on the position of an occluded segment of an artery blood vessel;

S2: obtaining an estimated length of an accompanying vein segment to be traversed based on the position information;

S3: calculating an injection speed and dosage of a contrast agent, so as to optimize a contrast agent injection plan and minimize an amount of the contrast agent to be used; and

S4: delivering a guide wire from an entrance position of the accompanying vein to a target position, accompanied by the injection of the contrast agent.

In one or more embodiments, the method further comprises a step S5 of: delivering an intravascular ultrasound (IVUS) to the accompanying vein along the guide wire.

In one or more embodiments, the method further comprises a step S6 of: operating the IVUS to guide travelling of the intra-arterial guide wire in a true lumen in real time by the IVUS.

In one or more embodiments, the target position is a distal end of the accompanying vein or a position in the accompanying vein corresponding to the occluded segment of the artery blood vessel.

In one or more embodiments, the step S3 further comprises a step of dividing the estimated length into a plurality of segments and calculating the injection speed and dosage of the contrast agent required for the individual segments.

In one or more embodiments, in the step S3, the calculation step further comprises steps of acquiring a blood flow velocity and blood pressure in the accompanying vein; calculating the injection speed and dosage of the contrast agent required for the segment based on the length of the current segment and based on the blood flow velocity and blood pressure; and adjusting an extension direction of the guide wire in the current segment in real time based on the actual extension direction of a previous segment of the accompanying vein.

In one or more embodiments, the artery is coronary artery, superficial femoral artery, internal carotid artery, or renal artery; and the accompanying vein is coronary vein, superficial femoral vein, internal jugular vein, or renal vein.

In one or more embodiments, the coronary artery is the anterior descending branch; the coronary vein is the great cardiac vein; and the entrance position is at the ostium of the coronary sinus.

In one or more embodiments, a process of injection of the contrast agent is associated with ON/OFF of a fluoroscopy device, so that they are synchronized with each other so as to reduce the intraoperative radiation dose.

In one or more embodiments, the process of injection of the contrast agent is associated with a cardiac cycle, and the injection of the contrast agent is controlled by means of cardiac cycle gating, blood pressure gating, or flow velocity gating. The injection is preferably implemented at a timing at which the blood flow velocity or blood pressure in the accompanying vein is the lowest.

In one or more embodiments, before the injection, sound and light reminders are provided for the cardiac cycle, and a countdown reminder is performed for cardiac cycles selected for the injection.

In one or more embodiments, the selected cardiac cycles are N cardiac cycles after an interaction signal is received from the user, so that a start timing of performing a process of delivery of the guide wire is determined by the user, where N is a natural number greater than or equal to 2. The interaction signal comes from a voice of the user, or is a signal from other input device (a mouse, a keyboard, or a pedal).

The present disclosure further provides an accompanying vein guiding system, comprising a guiding catheter for the accompanying vein, a guide wire, and a sensor component,

wherein the guiding catheter is configured to introduce the guide wire into the accompanying vein; and

the sensor component is configured to capture an image of an occluded segment of an artery blood vessel and obtain a blood flow velocity in the accompanying vein.

In one or more embodiments, the sensor component comprises an IVUS disposed at a distal end, and a flow velocity monitoring unit.

In one or more embodiments, the flow velocity monitoring unit is a flow meter or a Doppler module disposed more distally than the IVUS, and the flow meter is disposed at a distal end of the guide wire or at a distal end of the guiding catheter.

In one or more embodiments, the system further comprises a processor unit, configured to calculate an injection speed and dosage of a contrast agent based on the obtained estimated length of an accompanying vein segment to be traversed and based on the blood flow velocity obtained by the sensor component, so as to optimize a contrast agent injection plan and minimize an amount of the contrast agent to be used.

In one or more embodiments, the sensor component further comprises a pressure sensor to monitor blood pressure in the accompanying vein.

In one or more embodiments, the processor unit is configured to associate a process of injection of the contrast agent with ON/OFF of a fluoroscopy device so that they are synchronized with each other so as to reduce the intraoperative radiation dose.

In one or more embodiments, the processor unit further associates the injection process with a cardiac cycle and controls the injection of the contrast agent by means of cardiac cycle gating, blood pressure gating, or flow velocity gating. The injection is preferably implemented at a timing at which the blood flow velocity or blood pressure in the accompanying vein is the lowest.

In one or more embodiments, the processor unit further provides a reminder of the cardiac cycle by means of sound and light, and performs a countdown reminder for cardiac cycles selected for the injection before the injection.

In one or more embodiments, the processor unit sets the selected cardiac cycles as N cardiac cycles after an interaction signal is received from the user, so that a start timing of performing a process of delivery of the guide wire is determined by the user, where N is a natural number greater than or equal to 2. The interaction signal comes from a voice of the user, or is a signal from other input device (a mouse, a keyboard, or a pedal).

The accompanying vein guiding method and system according to the present disclosure include, for example, the following advantageous effects.

The IVUS placed in an accompanying vein of an artery blood vessel can capture a three-dimensional image of proximal and distal fibrous caps and an occluded segment so that their cross-sections are visible. In this way, when guiding the arterial guide wire, the three-dimensional position of the head end of the guide wire can be determined, which can avoid mistaken insertion of the head end into the dissection when the guide wire punctures the proximal or distal fibrous caps or travels in the occluded segment, thereby increasing the safety and success rate of the operation and resulting in a lower risk of intraoperative perforation of the coronary artery than that in a conventional operation.

Information on the position of the occlusion is automatically acquired by using the image information. The processor estimates the travel length of the guide wire in the accompanying vein and automatically gives an optimum contrast agent injection plan (whether to segment the estimated length, the injection speed/pressure), in order to minimize the use of the contrast agent. Compared to a conventional operation in which a large amount of a contrast agent (300 to 800 ml) is required, the accompanying vein guiding method of the present disclosure allows the amount of the contrast agent to be reduced to 70 ml or less, which is lower than the upper limit amount (100 ml) of a contrast agent tolerable by a patient with renal insufficiency. In this way, no occurrence of contrast-induced nephropathy is ensured under continuous monitoring of renal function after the operation, and the side effects and subsequent effects of the contrast agent on the patient are greatly reduced.

The operation takes only about 2 hours, which is much shorter than the duration of a conventional method (which lasts 3 to 4 hours or even longer).

The intraoperative radiation dose is significantly reduced compared with a conventional operation, thereby reducing the harm to medical staff and patients.

Moreover, fewer guide wires are used in the operation than in a conventional operation, thereby reducing the operation cost and alleviating the economic burden on patients.

### BRIEF DESCRIPTION OF THE DRAWINGS

In order to more clearly illustrate the technical solutions of the embodiments of the present disclosure or in the prior art, brief description is made below on the drawings required to be used in the description of the embodiments or the prior art. Obviously, the drawings described below illustrate only some of the embodiments of the present disclosure, and for a person of ordinary skills in the art, other drawings may be obtained from these drawings without inventive effort.

FIG. 1 is a schematic diagram of a coronary vein guiding catheter provided by the present disclosure;

FIG. 2 is a schematic structural diagram of the catheter wall of the coronary vein guiding catheter provided by the present disclosure;

FIG. 3 is a schematic diagram of a flexible inner tube provided by the present disclosure;

FIG. 4 is a flowchart of an accompanying vein guiding method according to the present disclosure;

FIG. 5 is a schematic diagram of a great cardiac vein guiding method according to the present disclosure;

FIG. 6 is a schematic structural diagram of a sensor component according to the present disclosure; and

FIG. 7 is a schematic operational diagram of the sensor component according to the present disclosure.

### REFERENCE SIGNS

coaxial section-1;
flexible head end-101,
anti-bending section-2;
torsion control section-3;
smooth surface layer-401;
elastic steel wire layer-402;
flexible outer
layer-403;
screw joint-501; blunt head end-502.

### DETAILED DESCRIPTION OF THE EMBODIMENTS

The technical solutions of the present disclosure will be clearly and completely described below in connection with the embodiments with reference to the drawings. However, it would be understood by a person skilled in the art that the described embodiments below are merely some of the embodiments of the present disclosure, rather than all the embodiments of the present disclosure, and are only used to explain the present disclosure, rather than limit the scope of the present disclosure. Based on the embodiments of the present disclosure, all the other embodiments obtained by a person of ordinary skills in the art without inventive efforts shall be covered by the scope of protection of the present disclosure. For the embodiments in which no specific conditions are given, the conventional conditions or the conditions recommended by manufacturers are used. The reagents or instruments, the manufacturers of which are not indicated, are all normal products commercially available.

In the description of the present disclosure, it is to be noted that the orientation or position relation denoted by the terms such as “center”, “upper”, “lower”, “left”, “right”, “vertical”, “horizontal”, “inner” and “outer” is based on the orientation or position relation indicated by the figures, which only serves to facilitate describing the present disclosure and simplify the description, rather than indicating or suggesting that the device or element referred to must have a particular orientation, and must be constructed and operated in a particular orientation. Therefore, said terms cannot be construed as limitations on the present disclosure. In addition, the terms “first”, “second” and “third” are only used for the description purpose, but cannot be construed as an indication or suggestion of relative importance.

In the description of the present disclosure, it should be noted that unless otherwise explicitly specified and defined, the terms “install”, “link” and “connect” shall be understood in broad sense, which may, for example, refer to fixed connection, detachable connection or integral connection; may refer to mechanical connection or electrical connection; may refer to direct connection or indirect connection by means of an intermediate medium; and may refer to communication between two elements. A person of ordinary skills in the art could understand the specific meaning of the terms in the present disclosure according to specific situations.

The present disclosure relates to a coronary vein guiding system, comprising a coronary vein guiding catheter, and a flexible inner tube sheathed into the coronary vein guiding catheter.

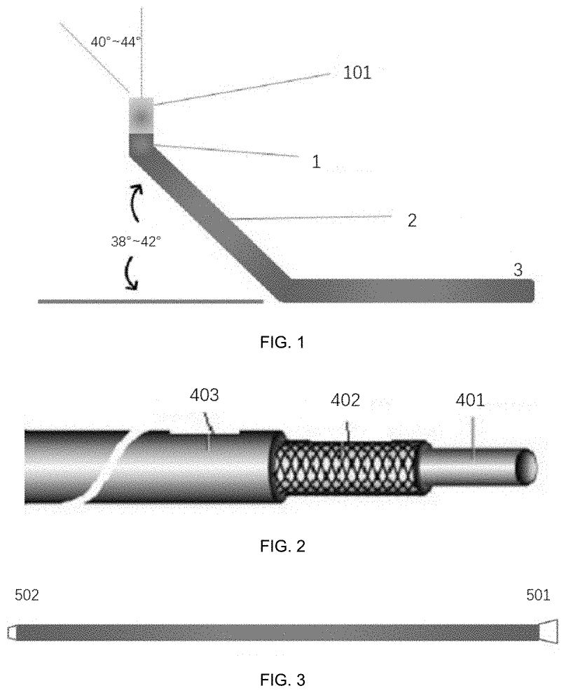

As shown in FIG. 1, the coronary vein guiding catheter sequentially comprises a coaxial section 1, an anti-bending section 2 and a torsion control section 3.

An end portion of the coaxial section 1 is provided with a flexible head end 101.

The coaxial section 1 is connected with the anti-bending section 2 at an obtuse angle of 136° to 140°.

The anti-bending section 2 is connected with the torsion control section 3 at an obtuse angle of 138° to 142°.

The coaxial section 1 and the torsion control section 3 are situated on a same side of the anti-bending section 2, and the three sections are located on a same plane.

As shown in FIG. 3, the flexible inner tube is 18 cm to 22 cm longer than the coronary vein guiding catheter.

A head end of the flexible inner tube is in the same direction as the coaxial section, and a tail end of the flexible inner tube is provided with a screw joint 501.

Preferably, the head end of the flexible inner tube is a blunt head end 502.

Preferably, the flexible inner tube has a two-layered tube wall, with an inner layer being a PVP hydrophilic coating and an outer layer being a HDPE high-density polyethylene.

Preferably, the screw joint is made of a PC material.

The screw joint 501 can be used for the connection with an external valve, such that the interior of the flexible inner tube is a vacuum, preventing blood overflowing or air entering.

Preferably, the total length of the flexible inner tube is 125 cm-135 cm (inserted from inferior vena cava (femoral vein)) or 60 cm-70 cm (inserted from superior vena cava (subclavian vein or jugular vein)).

More preferably, the total length of the flexible inner tube is 130 cm or 65 cm.

Preferably, the coaxial section 1 is connected with the anti-bending section 2 at an obtuse angle of 137° to 139°, most preferably, at an obtuse angle of 138°.

Preferably, the anti-bending section 2 is connected with the torsion control section 3 at an obtuse angle of 139° to 141°, most preferably, at an obtuse angle of 140°.

The coronary vein guiding catheter provided by the present disclosure is provided with two bending angles according to the anatomical features of heart so as to facilitate the insertion into the coronary sinus, wherein the first angle of 137° to 139° helps the coronary vein guiding catheter to enter the coronary vein, and the second angle of 139° to 141° enables the coronary vein guiding catheter to be fixed in the coronary sinus without dislocation.

After a transfer track is established by placing the coronary vein guiding catheter in the coronary sinus, a guide wire having ultrasound or other scanning imaging functions is taken to the great cardiac vein, and is located at occlusions in the anterior descending branch or the circumflex branch, by collecting information and uploading the same to a workstation, a three-dimensional image of the CTO lesion is reconstructed to guide the intracoronary guide wire to travel in a true lumen in real time, within the CTO occlusion.

Preferably, in order to prevent the coronary vein guiding catheter causing injuries to the interior of heart, the bending angle is in arc transition.

The coronary vein guiding catheter enters from the subclavian vein, the jugular vein or the femoral vein, and reaches the right atrium under the guidance of the guide wire, then the torsion control section 3 is twisted such that the flexible head end 101 is inserted into the coronary sinus; then the inner tube sticks out of the coronary vein guiding catheter under the assistance of the guide wire and travels in the great cardiac vein.

The coronary vein guiding system provided by the present disclosure can be used to guide the insertion of an ultrasound catheter having ultrasound or other scanning imaging functions into the coronary sinus, and the coronary vein guiding catheter can greatly reduce the difficulty in inserting the guide wire, reduce the technical requirements on the operator, and prevent the case that the ultrasound catheter cannot pass or cause adverse consequences such as perforation.

The ultrasound catheter may preferably be selected from the IVUS catheters. The ultrasound catheter in the veins can guide, by means of imaging, the doctors to observe the position of the working guide wire for dredging the CTO occlusion segments in an intervention operation, so as to achieve the object of eliminating a CTO lesion.

In addition, the coronary vein guiding system provided by the present disclosure can further be used for establishing a drug delivery channel to inject drugs to heart, for example, injecting nitroglycerin so as to achieve the object of dilating the blood vessels;
or for drawing blood through the channel established by the coronary vein guiding system, for example, drawing blood from the coronary vein via a catheter for a blood gas analysis test to determine the lactic acid level, so as to more accurately assess the myocardial metabolism level.

When the guide wire and the blood drawing or drug delivery device reach the designated position, the inner tube can be withdrawn.

Preferably, in the coronary vein guiding system described above, as shown in FIG. 2, the coronary vein guiding catheter comprises, from inside to outside, a smooth surface layer 401, an elastic steel wire layer 402 and a flexible outer layer 403 in this order.

More preferably, the flexible head end 101 only comprises a flexible outer layer 403.

The flexible head end 101 serves to prevent scratching of the coronary sinus, while the flexible inner tube serves to incur an elastic deformation when travelling in the coronary vein guiding catheter to enable entrance in a bent manner.

In the coronary vein guiding system described above, the smooth surface layer 401 serves to facilitate the travelling of the flexible inner tube, and prevent blocking of the flexible inner tube caused by the coronary vein guiding catheter itself.

Preferably, the smooth surface layer 401 is a nylon polytetrafluoroethylene coating. Polytetrafluoroethylene is abbreviated as PTFE, and products of such material are generally referred to as “non-stick coatings”/“materials for non-stick fry pans”. PTFE has the minimum friction coefficient among plastics, and is an ideal oil-free lubrication material.

In the coronary vein guiding system described above, the elastic steel wire layer 402 serves to provide rigidity and strength for the coronary vein guiding catheter on the one hand, and provide elasticity for the coronary vein guiding catheter on the other hand, such that the coronary vein guiding catheter can travel in the veins and in the right atrium.

Preferably, the elastic steel wire layer 402 can be a spring-shaped spiral structure, and more preferably, the elastic steel wire layer 402 is meshy woven steel wire.

More preferably, the elastic steel wire layer 402 is composed of 12 to 16 layers of steel wires.

In the coronary vein guiding system described above, the flexible outer layer 403 serves to wrap the elastic steel wire layer 402, to prevent scratching of the myocardial tissues, and the flexible outer layer 403 shall be made of the materials having good biocompatibility and no toxicity. The flexible head end 101 is also composed of a flexible outer layer 403, but for the sake of insertion into the venous sinus and travelling of the inner tube in the great cardiac vein, the flexible head end 101 also needs to have certain strength, preferably, the flexible outer layer is made of one or more of polyethylene, silica gel, polymethyl methacrylate, polyvinyl chloride, polytetrafluoroethylene, polyurethane, polyester polymers and silicone.

Preferably, in the coronary vein guiding system described above, the inner diameter of the coronary vein guiding catheter is 1.5 mm to 1.9 mm, and the thickness of the catheter wall is 0.15 mm to 0.35 mm.

More preferably, the inner diameter of the coronary vein guiding catheter is 1.6 mm to 1.8 mm, and the thickness of the catheter wall is 0.2 mm to 0.3 mm.

More preferably, the inner diameter of the coronary vein guiding catheter is 1.7 mm, and the thickness of the catheter wall is 0.25 mm.

Preferably, the inner diameter of the flexible inner tube is 1.25 mm to 1.4 mm, and the thickness of the tube wall of the flexible inner tube is 0.1 mm to 0.2 mm.

More preferably, the inner diameter of the flexible inner tube is 1.3 mm to 1.35 mm, and the thickness of the tube wall of the flexible inner tube is 0.15 mm.

The coronary vein guiding catheter and the flexible inner tube can be modified to have different types of inner diameters and thicknesses according to the sizes of the hearts of different patients.

Preferably, in the coronary vein guiding system described above, the length of the coaxial section 1 is smaller than the inner diameter of the right atrium of the inserted heart.

It shall be sufficient that the coaxial section 1 has a length that is smaller than the inner diameter of the right atrium, so as to be allowed to twist in the right atrium and arrive at the coronary sinus.

Preferably, in the coronary vein guiding system described above, the length of the anti-bending section 2 is smaller than the distance from the right atrium of the inserted heart to the coronary sinus; more preferably, the length of the anti-bending section 2 is 70 mm to 80 mm, or can be 75 mm.

Preferably, in the coronary vein guiding system described above, the length of the flexible head end 101 is 8 mm to 12 mm; or can be 9 mm to 11 mm; or 10 mm.

During the use of the system, the specific position of the catheter and the inner tube can be determined by in vitro injection of a contrast agent.

Preferably, in the coronary vein guiding system described above, if the method of using a contrast agent is not employed, it is also feasible to provide an indication section on the coaxial section 1 and provide a flexible inner tube indication section at the head end of the flexible inner tube, so as to indicate the position of the catheter and the inner tube.

The indication section and the flexible inner tube indication section are made of gold or platinum.

Preferably, the indication section on the coaxial section is connected with the flexible head end 101.

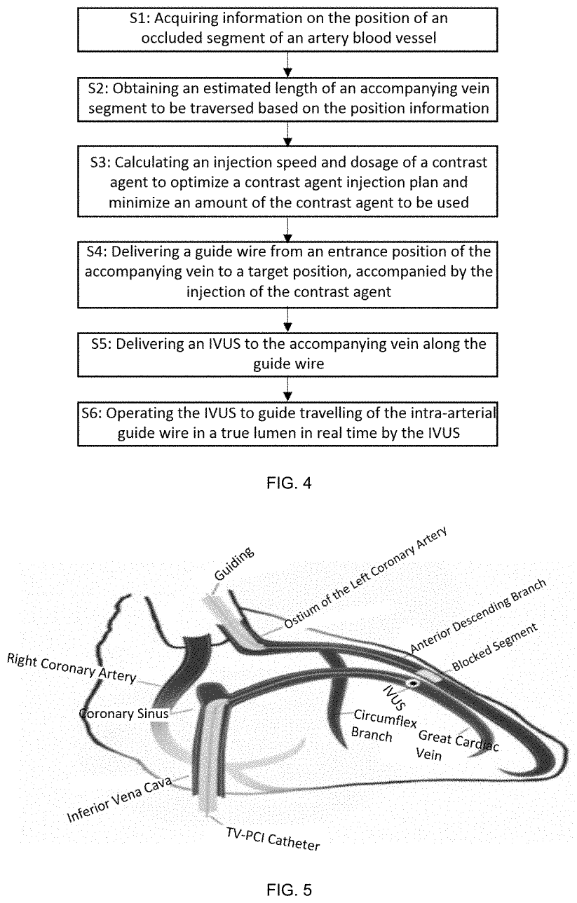

As shown in FIG. 4, the present disclosure also relates to an accompanying vein guiding method, comprising the steps of:

S1: acquiring information on the position of an occluded segment of an artery blood vessel;

S2: obtaining an estimated length of an accompanying vein segment to be traversed based on the position information;

S3: calculating an injection speed and dosage of a contrast agent to optimize a contrast agent injection plan and minimize an amount of the contrast agent to be used;

S4: delivering a guide wire from an entrance position of the accompanying vein to a target position, accompanied by the injection of the contrast agent;

S5: delivering an IVUS to the accompanying vein along the guide wire; and

S6: operating the IVUS to guide travelling of the intra-arterial guide wire in a true lumen in real time by the IVUS.

Here, the target position is a distal end of the accompanying vein or a position in the accompanying vein corresponding to the occluded segment of the artery blood vessel.

Further, the step S3 further comprises a step of dividing the estimated length into a plurality of segments and calculating the injection speed and dosage of the contrast agent required for the individual segments. Specifically, the estimated length may be divided into N segments according to different anatomical structures of the accompanying veins. For example, N is any positive integer from 1 to 8. Moreover, the calculation step further comprises steps of acquiring a blood flow velocity and blood pressure in the accompanying vein; calculating the injection speed and dosage of the contrast agent required for the segment based on the length of the current segment and based on the blood flow velocity and blood pressure; and adjusting an extension direction of the guide wire in the current segment in real time based on the actual extension direction of a previous segment of the accompanying vein.

The artery is coronary artery, superficial femoral artery, internal carotid artery, or renal artery; and the accompanying vein is coronary vein, superficial femoral vein, internal jugular vein, or renal vein. Particularly, the coronary artery is the anterior descending branch; the coronary vein is the great cardiac vein; and the entrance position is at the ostium of the coronary sinus.

Here, a process of injection of the contrast agent is associated with ON/OFF of a fluoroscopy device so that they are synchronized with each other so as to reduce the intraoperative radiation dose.

In addition, the process of injection of the contrast agent is associated with a cardiac cycle, and the injection of the contrast agent is controlled by means of cardiac cycle gating, blood pressure gating, or flow velocity gating. The injection is preferably implemented at a timing at which the blood flow velocity or blood pressure in the accompanying vein is the lowest, for example, in a period from the diastole to pre-systole of the left ventricle.

In addition, before the injection, sound and light reminders are provided for the cardiac cycle, and a countdown reminder is performed for cardiac cycles selected for the injection to remind the user to prepare for the delivery of the guide wire.

The selected cardiac cycles are N cardiac cycles after an interaction signal is received from the user, so that a start timing of preforming a process of delivery of the guide wire is determined by the user, where N is a natural number greater than or equal to 2. The interaction signal comes from a voice of the user, or is a signal from other input device (a mouse, a keyboard, or a pedal). The method of the present disclosure is described in further detail below according to a specific example.

### EXAMPLE

FIG. 5 shows a schematic diagram of a great cardiac vein guiding method in a transfemoral vein-percutaneous coronary intervention (TV-PCI).

Patient Information

The case was a male patient, with total (100%) occlusion in the middle segment of the anterior descending branch. A surgical operation would be extremely high-risk, and the patient strongly refused surgical bypass treatment. The patient suffered from renal insufficiency, and thus a contrast agent should be used in a limited amount. Therefore, TV-PCI was the best choice for the patient.

Operation Procedure

(1) Establishment of Approach: the right femoral vein was punctured, and a TV-PCI catheter was delivered to the ostium of the coronary sinus through the approach. The right radial artery was punctured, and a guiding catheter was delivered to the ostium of the left coronary artery through the approach. This step was the same as a conventional operation.

(2) The guiding catheter was delivered to the ostium of the left coronary artery through the radial artery approach, and then the guide wire is delivered to the occluded segment of the anterior descending branch through the guiding catheter.

(3) An estimated length of an accompanying vein segment (including the femoral vein, the inferior vena cava, and the great cardiac vein) to be traversed was estimated, and the accompanying vein segment was divided into three segments according to their different anatomical structures, and the blood flow velocity and/or blood pressure in each segment was measured at the entrance of the catheter or guide wire in each segment, so as to calculate the optimum speed and minimum dosage for injection of a contrast agent.

(4) Accompanied by the injection of the contrast agent, the TV-PCI catheter was delivered to the ostium of the coronary sinus through the femoral vein approach, and then the guide wire was delivered to the distal segment of the great cardiac vein through the guiding catheter.

(5) An IVUS was delivered to the great cardiac vein along the guide wire.

(6) The IVUS was operated to guide travelling of the guide wire in the anterior descending branch in the true lumen of the anterior descending branch in real time by the IVUS, and the angiography indicated that the guide wire reached the true lumen of the distal segment, whereby the operation was successfully completed.

Operation Effect

(1) The patient had a chronic occlusion in the anterior descending branch. A large amount of a contrast agent (300 to 800 ml) was required in a conventional operation, while only 70 ml of a contrast agent was used in the operation using the method according to the present disclosure.

(2) The patient suffered from renal insufficiency, and thus the amount of the contrast agent used should be controlled within 100 ml. Only 70 ml of the contrast agent was used during the entire operation, and no contrast-induced nephropathy was observed under continuous monitoring of renal function after the operation.

(3) It took a long time of generally 3 to 4 hours or even longer to unblock the blood vessel by a conventional method, while the entire operation procedure according to the method provided in the present disclosure lasted only about 2 hours.

(4) The intraoperative radiation dose was significantly reduced compared with a conventional operation.

(5) There was a lower risk of intraoperative perforation of the coronary artery than that in a conventional operation.

(6) Fewer guide wires were used in the operation than in a conventional operation, and the cost was reduced.

The present disclosure also relates to an accompanying vein guiding system, comprising a guiding catheter, a guide wire, a sensor component and a processor unit for the accompanying vein. The guiding catheter is configured to introduce the guide wire into the accompanying vein. The sensor component is configured to capture an image of an occluded segment of an artery blood vessel and obtain a blood flow velocity in the accompanying vein.

Here, the sensor component comprises an IVUS disposed at a distal end, and a flow velocity monitoring unit. Here, the flow velocity monitoring unit is a flow meter or a Doppler module disposed more distally than the IVUS, and the flow meter is disposed at a distal end of the guide wire or at a distal end of the guiding catheter.

The processor unit is configured to calculate an injection speed and dosage of a contrast agent based on the obtained estimated length of an accompanying vein segment to be traversed and based on the blood flow velocity obtained by the sensor component to optimize a contrast agent injection plan and minimize an amount of the contrast agent to be used.

In addition, the sensor component further comprises a pressure sensor to monitor blood pressure in the accompanying vein.

The processor unit is configured to associate a process of injection of the contrast agent with ON/OFF of a fluoroscopy device so that they are synchronized with each other so as to reduce the intraoperative radiation dose.

In addition, the processor unit further associates the injection process with a cardiac cycle and controls the injection of the contrast agent by means of cardiac cycle gating, blood pressure gating, or flow velocity gating. The injection is preferably implemented at a timing at which the blood flow velocity or blood pressure in the accompanying vein is the lowest, for example, in a period from the diastole to pre-systole of the left ventricle.

In addition, the processor unit further provides a reminder of the cardiac cycle by means of sound and light, and performs a countdown reminder for cardiac cycles selected for the injection before the injection so as to remind the user to prepare for the delivery of the guide wire.

The processor unit sets the selected cardiac cycles as N cardiac cycles after an interaction signal is received from the user, so that a start timing of performing a process of delivery of the guide wire is determined by the user, where N is a natural number greater than or equal to 2. The interaction signal comes from a voice of the user, or is a signal from other input device (a mouse, a keyboard, or a pedal).

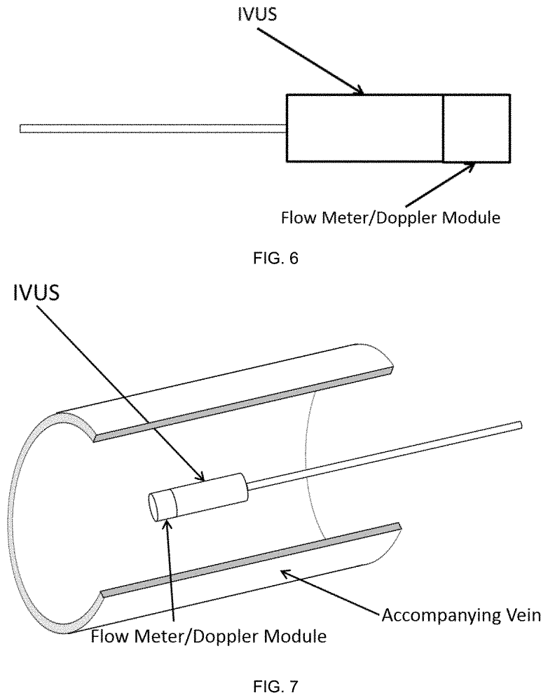

Referring to FIGS. 6 and 7, the sensor component comprises an IVUS disposed at a distal end, and a flow velocity monitoring unit. The flow velocity monitoring unit is a flow meter or a Doppler module disposed more distally than the IVUS, and the flow meter is disposed at a distal end of the guide wire or at a distal end of the guiding catheter.

Finally, it should be noted that the above embodiments are only used to illustrate the technical solutions of the present disclosure, rather than limiting the same; although the present disclosure has been described in detail with reference to the foregoing embodiments, it should be understood by a person of ordinary skills in the art that the technical solutions described in the embodiments can still be modified, or equivalent substitution can be made to some or all of the technical features therein; and the modification or substitution would not cause the substance of the corresponding technical solutions to get out of the scope of the technical solutions of the embodiments of the present disclosure.

### INDUSTRIAL APPLICABILITY

In the accompanying vein guiding method and system according to the present disclosure, The IVUS placed in a vein accompanying an artery blood vessel can capture a three-dimensional image of proximal and distal fibrous caps and an occluded (or occlusion) segment so that their cross-sections are visible. In this way, when guiding the arterial guide wire, the three-dimensional position of the head end of the guide wire can be determined, which can avoid mistaken insertion of the head end into the dissection when the guide wire punctures the proximal or distal fibrous caps or travels in the occluded segment, thereby increasing the safety and success rate of the operation and resulting in a lower risk of intraoperative perforation of the coronary artery than that in a conventional operation. Information on the position of the occlusion is automatically acquired by using the image information. The processor estimates the travel length of the guide wire in the accompanying vein and automatically gives an optimum contrast agent injection plan (whether to segment the estimated length, the injection speed/pressure), in order to minimize the use of the contrast agent. Compared to a conventional operation in which a large amount of a contrast agent (300 to 800 ml) is required, the accompanying vein guiding method of the present disclosure allows the amount of the contrast agent to be reduced to 70 ml or less, which is lower than the upper limit amount (100 ml) of a contrast agent tolerable by a patient with renal insufficiency. In this way, no occurrence of contrast-induced nephropathy is ensured under continuous monitoring of renal function after the operation, and the side effects and subsequent effects of the contrast agent on the patient are greatly reduced. The operation takes only about 2 hours, which is much shorter than the duration of a conventional method (which lasts 3 to 4 hours or even longer). The intraoperative radiation dose is significantly reduced compared with a conventional operation, thereby reducing the harm to medical staff and patients. Moreover, fewer guide wires are used in the operation than in a conventional operation, thereby reducing the operation cost and alleviating the economic burden on patients.

What is claimed is:

1. An accompanying vein guiding method, comprising following steps of:
S1: acquiring position information of an occluded segment of an artery blood vessel;
S2: obtaining an estimated length of an accompanying vein segment to be traversed based on the position information;
S3: calculating an injection speed and a dosage of a contrast agent, so as to optimize a contrast agent injection plan and minimize an amount of the contrast agent to be used; and
S4: delivering a guide wire from an entrance position of the accompanying vein to a target position, accompanied by the injection of the contrast agent.

2. The accompanying vein guiding method according to claim 1, further comprising a step S5 of: delivering an intravascular ultrasound to the accompanying vein along the guide wire.

3. The accompanying vein guiding method according to claim 2, further comprising a step S6 of: operating the intravascular ultrasound to guide an intra-arterial guide wire to travel in a true lumen in real time by the intravascular ultrasound.

4. The accompanying vein guiding method according to claim 1, wherein the target position is a distal end of the accompanying vein or a position in the accompanying vein corresponding to the occluded segment of the artery blood vessel.

5. The accompanying vein guiding method according to claim 1, wherein the step S3 further comprises dividing the estimated length into a plurality of segments and calculating the injection speed and the dosage of the contrast agent required for each of the segments.

6. The accompanying vein guiding method according to claim 5, wherein in the step S3, the calculating further comprises acquiring a blood flow velocity and blood pressure in the accompanying vein; calculating the injection speed and the dosage of the contrast agent required for a current segment based on a length of the current segment and based on the blood flow velocity and blood pressure; and adjusting an extension direction of the guide wire in the current segment in real time based on an actual extension direction of a previous segment of the accompanying vein.

7. The accompanying vein guiding method according to claim 1, wherein the artery is coronary artery, superficial femoral artery, internal carotid artery or renal artery; and the accompanying vein is coronary vein, superficial femoral vein, internal jugular vein or renal vein.

8. The accompanying vein guiding method according to claim 7, wherein the coronary artery is anterior descending branch; the coronary vein is great cardiac vein; and the entrance position is at an ostium of coronary sinus.

9. The accompanying vein guiding method according to claim 1, wherein a process of the injection of the contrast agent is associated with ON/OFF of a fluoroscopy device, so that they are synchronized with each other so as to reduce intraoperative radiation dose; the process of the injection of the contrast agent is associated with cardiac cycles, the injection of the contrast agent is controlled by means of cardiac cycle gating, blood pressure gating, or flow velocity gating, and the injection is implemented at a timing at which the blood flow velocity or blood pressure in the accompanying vein is the lowest; before the injection, sound and light reminders are provided for the cardiac cycles, and a countdown reminder is performed for cardiac cycles selected for the injection, so as to remind a user to prepare for delivering the guide wire, wherein selected cardiac cycles are N cardiac cycles after an interaction signal is received from the user, so that a start timing of performing a process of delivering the guide wire is determined by the user, where N is a natural number greater than or equal to 2, wherein the interaction signal comes from a voice from the user, or is a signal input from a mouse, a keyboard or a pedal.

10. An accompanying vein guiding system, comprising a guiding catheter, a guide wire and a sensor component for the accompanying vein,
wherein the guiding catheter is configured to introduce the guide wire into the accompanying vein; and
the sensor component is configured to capture an image of an occluded segment of an artery blood vessel and obtain a blood flow velocity in the accompanying vein.

11. The accompanying vein guiding system according to claim 10, wherein the sensor component comprises an intravascular ultrasound disposed at a distal end, and a flow velocity monitoring unit.

12. The accompanying vein guiding system according to claim 11, wherein the flow velocity monitoring unit is a flow meter or a Doppler module disposed more distally than the intravascular ultrasound, and the flow meter is disposed at a distal end of the guide wire or at a distal end of the guiding catheter.

13. The accompanying vein guiding system according to claim 10, further comprising a processor unit, configured to calculate an injection speed and a dosage of a contrast agent based on an obtained estimated length of an accompanying vein segment to be traversed and based on the blood flow velocity obtained by the sensor component, so as to optimize a contrast agent injection plan and minimize an amount of the contrast agent to be used.

14. The accompanying vein guiding system according to claim 10, wherein the sensor component further comprises a pressure sensor, so as to monitor a blood pressure in the accompanying vein.

15. The accompanying vein guiding system according to claim 13, wherein the processor unit is configured to associate a process of the injection of the contrast agent with ON/OFF of a fluoroscopy device, so that they are synchronized with each other so as to reduce intraoperative radiation dose; the processor unit further associates a process of the injection with cardiac cycles and controls the injection of the contrast agent by means of cardiac cycle gating, blood pressure gating, or flow velocity gating; the injection is implemented at a timing at which the blood flow velocity or blood pressure in the accompanying vein is the lowest; the processor unit further provides a reminder of the cardiac cycles by means of sound and light, and performs a countdown reminder for cardiac cycles selected for the injection before the injection, so as to remind a user to prepare for delivering the guide wire, wherein the processor unit sets selected cardiac cycles as N cardiac cycles after an interaction signal is received from the user, so that a start timing of performing a process of delivering the guide wire is determined by the user, where N is a natural number greater than or equal to 2, wherein the interaction signal comes from a voice from the user, or is a signal input from a mouse, a keyboard or a pedal.
